(19) United States
(12) Patent Application Publication
(10) Pub. No.: US 2006/0014728 A1
(43) Pub. Date: Jan. 19, 2006

(54) Hormone replacement therapy

(76) Inventors: Kristof Chwalisz, Berlin (DE); Gary D. Hodgen, Norfolk, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/025,977

(22) Filed: Jan. 3, 2005

Related U.S. Application Data
(63) Continuation of application No. 09/395,982, filed on Sep. 15, 1999, now abandoned.
Application No. 09/395,982 is a continuation of application No. 08/752,948, filed on Nov. 21, 1996, now abandoned.

(51) Int. Cl. A61K 31/56

(52) U.S. Cl. 514/170

(57) ABSTRACT

A method of hormone replacement therapy comprises administering to a woman in need of such therapy, estrogen in an hormone replacement therapy effective amount and antiprogestin in an amount which is effective both to inhibit estrogen-induced endometrial proliferation and to effect a state of substantial amenorrhea, in the absence of progestin administration.

In the post-menopausal years, women often receive hormone replacement therapy. This can involve administration of estrogen and progestin or estrogen only. For women with a uterus, estrogen-only therapy has been associated with increased risk of endometrial cancer. To offset the involved estrogen-induced proliferation of the endometrium, progestin has been administered. However, the combination of estrogen and progestin has the undesirable side effect of uterine bleeding which can reduce the rate of patient compliance.

### SUMMARY OF THE INVENTION

This invention relates to a method of hormone replacement therapy (HRT) which comprises administering to a woman in need of such therapy, estrogen in an hormone replacement therapy effective amount and antiprogestin in an amount which is effective both to inhibit estrogen-induced endometrial proliferation and to effect a state of substantial amenorrhea, in the absence of progestin administration.

Suitable estrogens, amounts and regimens are in accordance with conventional considerations for HRT therapy. Examples of estrogens which can be employed in this invention are ethinyl estradiol and estradiol and their esters, e.g., acetate, valerate, benzoate and undecylate, mestranol and conjugated estrogens. Administration can be by any route, e.g., orally or transdermally. For example, the amount of conjugated equine estrogen administered is analogous to that practiced in conventional estrogen replacement therapy and is generally in the range of about 0.3 to 1.2 mg, preferably about 0.625 to 0.9 mg daily. The determination of an effective dose is routine, taking into account the usual physical parameters, such as weight, age and the like, and is best determined by the attending clinician. The administration can be periodic or continuous. The latter, e.g., daily administration, is preferred because individuals are more likely to follow the treatment regimen and not to forget or overlook a periodic administration schedule.

Suitable antiprogestins include progesterone receptor antagonists or inhibitors of the biological activity of progesterone, and the like. Examples of antiprogestins which can be employed in this invention are RU 486 (“Mifepristone,” Roussel Uclaf, Paris); and “Onapristone” (Schering A G, Berlin; U.S. Pat. No. 4,780,461) and the steroids described in the examples and the following patents and patent applications: U.S. Pat. No. 4,609,651, especially the compound lilopristone (11β-(4-dimethylamino-phenyl-17β-hydroxy-17α-(3-hydroxy-prop-1-(Z)-enyl-4,9(10) estradien-3-one); U.S. application Ser. No. 06/827,050, especially the compounds 11β-4-acetylphenyl)-17β-hydroxy-17α-(1-propinyl)-4,9-estradien-3-one and 11β-(4-acetylphenyl)-17β-hydroxy-17α-(3 hydroxy-1(2)-propenyl)-4,9-estradien-3-one; U.S. application Ser. No. 07/283,632; U.S. application Ser. No. 07/541,806, corresponding to published European patent application EP-A 04042831; and other antigestagens, e.g., U.S. Pat. No. 4,891,368. The antiprogestin can be administered by way of any art recognized means as practiced in the pharmaceutical arts. For example, a suitable antiprogestin may be formulated so that it can be administered orally, via a skin patch for transdermal absorption, contained within an inert matrix which is implanted within the body and in the depot state or intravaginally in a matrix that slowly releases the antiprogestin; (Such an implant is taught in U.S. Pat. Nos. 4,957,119 and 5,088,505 and the like.)

Suitable pharmaceutical formulations are highly conventional and disclosed, e.g., in the various references mentioned herein and elsewhere. The estrogen and antiprogestin components can be administered separately or in the same dosage form, e.g., tablet.

The pharmaceutical formulations may be provided in kit form containing a plurality of, generally at least about 20, and preferably in multiples of 7 such as 28, tablets, intended for ingestion on successive days. Where administration of the antiprogestin is intended to be periodic, a plurality, generally at least three, of non-adjacent tablets contain the antiprogestin while the remaining tablets are placebo. Where convenient, the kit may provide the estrogen and antiprogestin can be in the same tablet.

The antiprogestin can be administered essentially continuously or periodically, e.g., intermittently. The two types of regimens are equivalent. When the antiprogestin is administered intermittently, higher doses, of course, will be administered in comparison to a continuous antiprogestin regimen. Suitable intermittent dosages include 50-500 mg over 1-3 days, e.g., about 1-10 mg/kg. Administration periods can be weekly, biweekly, every 20 days, monthly, etc. For more continuous regimens, e.g., weekly or more frequently, e.g., daily, every other day, etc., amounts are generally about 0.005 to 1 mg/kg and preferably about 0.05 to 0.5 mg/kg, daily in the case of RU 486. Other milligram amounts may be appropriate in the case of different antiprogestins. Regimens of estrogen and antiprogestin, other than daily and/or in which the dosage amount of the estrogen and/or antiprogestin is periodically varied are also within the scope of the invention. Daily administration is preferred for ease of compliance. For RU 486, a suitable human oral dose would be on the order of about 0.5 to 10 mg per dose, preferably about 1 to 5 mg per dose daily. This amount can be lowered or increased based on the particular regimen utilized and the usual characteristics involved., e.g., the nature of the individual.

In all cases, the amount of antiprogestin administered will be an amount which inhibits estrogenic endometrial proliferation and enables achievement of amenorrhea. The amenorrhea state established by this invention is substantially, but not necessarily totally, complete. Thus, it will be appreciated that a minor amount of periodic bleeding or spotting on a monthly or yearly basis can occur.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and unless otherwise indicated, all parts and percentages are by weight.

The entire disclosure of all applications, patents and publications, cited above and below.

### EXAMPLES

### Example 1

30 ovariecomized cynomolgus monkeys were studied intensively over 12 weeks. Primates in Group I (N=5 each) received only the vehicles used for hormone delivery in Groups II-VI. Monkeys in Group II received estradiol at physiologic levels throughout (days 1 to 81) via a sc silastic implant, as well as two separate courses of progesterone at physiologic levels via a sc silastic implant (days 28 to 35 and 67 to 74). Groups III-VI received the same sequential regimen of estradiol and progesterone as Group II, but in addition were given an antiprogestin im on alternate days 39 to 67: Group III, mifepristone (RU 486) 2.0 mg/kg; Group IV, mifepristone 20.0 mg/kg; Group V, onapristone (ZK 299) 2.0 mg/kg; and Group VI, onapristone 20.0 mg/kg. The two menstrual challenge tests (A and B) were defined as the six days immediately after withdrawal of progesterone treatment (A on days 36 to 41 and B on days 75 to 80).

The protocol thus provided two periods of estrogen-only HRT treatment, one without antiprogestin followed by another with antiprogestin. Each of these two periods was followed by short-term progesterone treatment which provided a measurement of the effects on bleeding of the antiprogestin during the estrogen HRT treatment.

Additional data were collected by measurements of estradiol, progesterone and cortisol, as well as each antiprogestin in serum drawn on alternate days (1 to 81). Also, endometrial response was examined histologically in biopsy specimens collected on days 53 and 70, both to measure thickness of endometrial tissue (i.e., anti-proliferative response) and to assess the endometrial tissue qualitatively (atrophic, proliferative, or secretory, etc.).

Antiprogestin Compounds

Onapristone (ZK 299) (1β-(4-dimethylaminophenyl)-17-hydroxy-17β-(3-hydroxypropyl)-13-methyl-4,9-gonadien-3-one) and mifepristone (RU 486) (11β-(4-dimethylaminophenyl)-17β-hydroxy-1-propinyl-4,9estradien-3-one) were dissolved in benzyl benzoate, then mixed with castor oil (2.3, vol:vol). Both antiprogestins were synthesized at Schering A G (Berlin, Germany).

Primate Model

Thirty normally cycling adult female cynomolgus monkeys were ovariectomized at least 45 days prior to the initiation of this study.

The monkeys were randomly distributed into six treatment group preparations (n=5 each). Group I (control) was treated with the vehicle only. One empty silastic implant was placed sc surgically from study day 1 to 81; this controlled for estradiol treatment. Another empty silastic implant was inserted sc from days 28 to 35 and from days 67 to 74 and controlled for progesterone treatment. One ml of the antiprogestin vehicle was administered im on alternate days (39 to 67). Group II received continuous estradiol progesterone at two intervals but no antiprogestins. This experimental design allowed two menstrual challenges (A and B). As described below (Groups III-VI), menstrual challenge A was without antiprogestin exposure; whereas B was with antiprogestin treatment. Group III monkeys received the sequential estradiol and progesterone regimens as in Group II but also RU 486 at 2.0 mg/kg on alternate days for four weeks (days 39 to 67). For Group IV, the same treatment occurred, except that the RU 486 dose was elevated by ten-fold (20.0 mg/kg). Similarly, primates in Groups V and VI were injected with onapristone at 2.0 and 20.0 mg/kg, respectively.

Endometrial Biopsies and Tissue Characterization

In all treatment groups, observations for menstrual bleeding were made daily by visual inspection of the vaginal labia. Femoral blood collections (4.0 ml) were taken under ketamine-induced anesthesia (10 mg/kg) on alternate days and endometrial biopsies (by hysterotomy) were obtained on days 53 and 70. Endometrial biopsies were placed in formalin by staining and processed for histologic evaluation by a clinical pathologist. Thickness of the endometrial tissues was measured with an ocular micrometer to determine the depth of the endometrium at its point of maximal thickness from the myometrial unction to the epithelial layer at the lumenal surface. Endometrial status was evaluated qualitatively and classified according to the following score: atrophic:O; early proliferative: 1; late proliferative:2; interval:3; early secretory:4; and late secretory:5.

Steroid Determination

Blood samples were permitted to clot; serum was harvested and stored at 20° C. RIA's were performed for mifepristone, estradiol, progesterone and cortisol. Onapristone was quantified by HPLC with the detection limit of 4 ng/ml.

Statistical Analysis

Results were expressed as the mean+SD. Statistical comparisons were made. The results are shown in Tables 1 and 2.

The results illustrate that the incidence of withdrawal bleeding during the six days after removal of progesterone challenge in menstrual period A (estrogen(E)-only, no antiprogestin(AP)) was 31.3% (47 of 150 days, Groups II-VI combined), while zero among Group I primates (vehicle controls). For menstrual period B, again the Group I control Monkeys had no bleeding episodes. Also sustained was a 30.0% (9 of 30 days) bleeding incidence among estradiol plus progesterone only treated control monkeys (Group II). In contrast, E-only plus antiprogestin treatment (Groups III-VI) manifested only a 2.5% (3 of 120 days) combined incidence of withdrawal bleeding. These three incidents of induced menses all occurred in association with onapristone at the lower dose of 2.0 mg/kg. Using mifepristone at either 2.0 or 20.0 mg/kg, or onapristone at 20.0 mg/kg, uniformly led to complete amenorrhea during menstrual challenge B. These differences in bleeding rates for E-only HRT without versus with antiprogestin exposure are highly significant (P<0.01, combined groups).

Endometrial growth (thickness) at day 53 was approximately 2.5 mm in all treatment groups (II to VI), versus vehicle controls at about 0.5 mm. However, on day 70, among the therapeutic regimens of Groups III-VI, thicknesses of endometrial

TABLE I
Incidence of withdrawal menses^{a} after sequential estradiol^{b} and
progesterone^{c} therapy without versus with antiprogestin treatment^{d}

| | Menstrual Challenge A (Days) | | Menstrual Challenge B (Days) | |
|---|---|---|---|---|
| | Antiprogestins: None | | Antiprogestins: 39-67 | |
| | Progesterone: 28-35 | | Progesterone: 67-74 | |
| Experimental Design | Withdrawal interval: 36-41 | | Withdrawal Interval: 75-80 | |
| | incidence of Bleeding | | incidence of Bleeding | |
| Treatment Groups (N = 5) | Days | % | Days | % |

| | | | | | | | | | |
|---|---|---|---|---|---|---|---|---|---|
| I. Vehicle Control | 0/30 | | | 0 | 0/30 | | | 0 | |
| II. Estradiol + Progesterone only | 4/30 | | | | 9/30 | | | | 30 |
| III. Estradiol + Progesterone + MIF @ 2 mg/kg | 13/30 | | | | 0/30 | | | | |
| IV. Estradiol + Progesterone + MIF @ 20 mg/kg | 12/30 | {close oversize brace} | 47/150 | 31.3 | 0/30 | | 3/120 | 2.5 | |
| V. Estradiol + Progesterone + ONA @ 2 mg/kg | 9/30 | | | | 3/30 | {close oversize brace} | | | |
| VI. Estradiol +Progesterone + ONA @ 20 mg/kg | 9/30 | | | | 0/30 | | | | |

^{a}Menses is defined here to be two or more consecutive days of blood observed on the vaginal labia within six days after withdrawal of progsterone treatment.
^{b}Estradiol was administered via sc silastic implant throughout the study (Groups II-VI).
^{c}Progesterone was administered via sc silastic implant in two sequential regimens of seven days each.
^{d}Antiprogestions (MIFepristone and ONApristone) were injected im on alternate days 39-67 (Groups III-VI).

TABLE 2
Histologic evaluation of primate endometrial tissues on days 53 and 70

| | | | | | |
|---|---|---|---|---|---|
| Group I | Group II | Group III | Group IV | Group V | Group VI |

Day 53

| | | | | | | |
|---|---|---|---|---|---|---|
| Thickness (mm) | 0.4 ± 0.1 | 2.6 ± 0.1* | 2.5 ± 0.2* | 2.2 ± 0.2* | 2.6 ± 0.2* | 2.2 ± 0.1* |
| Endometrial | 0 | 4.0 | 3.6 | 3.8 | 3.8 | 3.6 |
| Score | | | | | | |
| Day 70 | | | | | | |
| Thickness (mm) | 0.5 ± 0.1 | 2.7 ± 0.1 | 1.8 ± 0.2** | 1.3 ± 0.1** | 1.6 ± 0.1** | 1.7 ± 0.1** |
| Endometrial | 0 | 4.3 | 2.4 | 2.8 | 4.0 | 3.0 |
| Score | | | | | | |

Score:
0-atrophic
1-proliferative
2-late proliferative
3-interval
4-early secretory
5-secretory
*Statistically significant differences (p < 0.05) from controls (Group I)
**Statistically significant differences (p < 0.05) from controls (Group I) and sequential estradioilprogesterone only (Group II)

tissues were significantly (P<0.05) diminished after exposure to the antiproliferative influences of RU 486 and onapristone (approximately 1.5 mm) compared to the absence of antiprogestin in Group II (2.7 mm). This effect was most profound in the high dose, RU 486 group (IV, 20.0 mg/kg). Upon histological evaluation, estradiol and progesterone alone had induced development of lush secretory endometrium present at both days 53 and 70 (Group II). Similarly, the low dose of onapristone (Group V, 2.0 mg/kg) manifested well-developed secretory glands, even though the tissue thickness was reduced (1.6 mm); this is the same group of monkeys in which only days (three) of withdrawal bleeding were observed after antiprogestin administration (menstrual challenge B). The other primates (Groups III, IV and VI) displayed differential endometrial states, ranging from late proliferative to early secretory characteristics, in association with tissue compaction (reduced endometrial thickness), as well as maintaining amenorrhea uniformly during menstrual challenge B.

These data show that E-only+continuous AP treatment was effective to achieve no breakthrough bleeding throughout the entire study, i.e., it induced a state of sustained amenorrhea and also displayed antiproliferative effects (weak) on the endometrium.

### Example 2

The purpose of this study was to evaluate the antiproliferative action of antiprogestins on the endometrium in estrogen-replaced, ovariectomized cynomolgus monkeys.

Previously ovariectomized cynomolgus monkeys had implanted subcutaneously a 3 cm estradiol containing silastic capsule on day 1 of the study. This capsule was removed on day 30. The vehicle or antiprogestin was administered intramuscularly on a daily basis from day 1 to 30. Vaginal swabs were performed daily from the first day of treatment. Femoral blood samples (3.5 cc) were collected under ketamine-induced anesthesia (10 mg/kg, im) on day 1, 8, 15, 22 and 30 for analyses of estradiol, cortisol and antiprogestin. An endometrial biopsy was obtained by hysterotomy on day 30 under ketamine (20 mg/kg)/xylazine (1 mg/kg) anesthesia. Nubain (1 mg/kg) was administered post-operatively for analgesia every twelve hours as needed. The endometrial biopsy was processed for histology; the specimens were classified by developmental stage, endometrial thickness, PCNA, K167, ER and PR.

The treatment groups, with three monkeys per group, were as follows:

1. Myrj—vehicle control, im

2. ZK136798 1 mg/kg, im

3. ZK136798 3 mg/kg, im

4. ZK137316 1 mg/kg, im

5. ZK137316 3 mg/kg, im

6. Onapristone 1 mg/kg, im

7. Onapristone 3 mg/kg, im

8. RU 486 1 mg/kg, im

9. RU 486 3 mg/kg, im

(ZK numbers are in-house designations of Schering, A G, Berlin, Germany.)

The endometrial specimens from monkeys treated with ZK137316 were primarily classified as “regressed-proliferative.” In contrast, the other groups were primarily classified as control—early secretory:

1. ZK136798—interval

2. Onapristone—interval

3. RU 486—proliferative

The specimens classified as “regressed proliferative” had proliferative characteristics but had reduced glandular size and dense stroma. The thickness of the endometrium from antiprogestin treated monkeys was less than that of the vehicle-treated control monkeys.

The number of mitotic spindles per ten glands was as follows:

1. control—2

2. Onapristone—0.7 to 1.0

3. ZK136798—0.5 to 1.0

4. RU 486—0.0 to 0.3

5. ZK137316—0.0

There were no meaningful differences between groups relative to PCNA labeling. PCNA activity and mitotic activity become disassociated with antiprogestin treatment. Of the antiprogestins evaluated in this continuous administration mode, ZK137316 appears to be the most effective in inhibiting endometrial proliferation.

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of hormone replacement therapy which comprises administering to a woman in need of such therapy, estrogen in an hormone replacement therapy effective amount and antiprogestin in an amount which is effective both to inhibit estrogen-induced endometrial proliferation and to effect a state of substantial amenorrhea, in the absence of progestin administration.

2. The method of claim 1 in which the antiprogestin is administered daily.

3. The method of claim 2 in which the administration is oral.

4. The method of claim 1 in which the estrogen and antiprogestin are administered daily.

5. The administration of claim 1 in which the administration is oral.

6. The method of claim 1 in which each administration contains about 0.5 to 10 mg of the antiprogestin daily.

7. The method of claim 6 in which the amount is about 1 to 5 mg.

8. The method of claim 1 in which the mode of administration is by depot.

9. The method of claim 1 in which the antiprogestin is a progestin receptor antagonist.

10. The method of claim 9 in which the antiprogestin is RU 486.

11. The method of claim 1 in which the administration extends over a minimum interval of 20 days.

12. In a method of hormone replacement therapy in which estrogen is administered in the absence of progestin administration to a woman in need of such therapy, the improvement which comprises the additional administration to said woman of antiprogestin in an amount which both inhibits estrogen-induced endometrial proliferation and effects a state of substantial amenorrhea

13. A kit containing at least 20 tablets, a portion of which contain a hormone replacement therapy effective amount of an estrogen and at least 20 of which contain an amount of an antiprogestin which both inhibits estrogen-induced endometrial proliferation and effects a state of substantial amenorrhea.

14. The kit of claim 13 in which each tablet contains both the estrogen and antiprogestin.

15. The kit of claim 14 in which the amount of antiprogestin is about 0.5 to 10 mg.

16. The kit of claim 15 in which the amount of antiprogestin is about 1 to 5 mg.

17. The kit of claim 16 in which the antiprogestin is RU 486.

18. The kit of claim 13 in which the amount of antiprogestin is about 0.5 to 10 mg.

19. The kit of claim 13 in which the amount of antiprogestin is about 1 to 5 mg.

20. The kit of claim 13 in which the antiprogestin is a progestin receptor antagonist.
